(19) United States
(12) Patent Application Publication
(10) Pub. No.: US 2015/0165033 A1
(43) Pub. Date: Jun. 18, 2015

(54) COMPOSITION FOR NASAL APPLICATION

(71) Applicant: BBU-VITA CORP., Virgin Islands (VG)

(72) Inventors: Dimitry Terterov, St. John's (CA); Sergei Terterov, Los Angeles, CA (US); Naum Goldstein, Berlin (DE); Roman Goldstein, Berlin (DE); Alexandr Terterov, Moscow (RU)

(21) Appl. No.: 14/399,773

(22) PCT Filed: May 6, 2013
(86) PCT No.: PCT/DE2013/000261

§ 371 Date: Mar. 4, 2015

(30) Foreign Application Priority Data
May 9, 2012 (DE) 10 2012 009 570.8

(51) Int. Cl. A61K 47/18; A61K 9/00; A61K 47/08; A61K 33/00

(52) U.S. Cl. CPC A61K 47/18; A61K 33/00; A61K 9/0043; A61K 47/08

(57) ABSTRACT

The invention relates to a pharmaceutical composition and arrangement thereof for nasal application. The aim of the invention is to increase the efficacy of drugs, metabolites and/or other previously described types of active ingredients that are indicated for the treatment of diseases and disorders of the central nervous system, to reduce the usually used dosage of the drugs, and to achieve a more rapid and prolonged effect. The pharmaceutical composition, composed of reciprocally related biologically active substances and substances that act on the nasal mucous membrane, is filled and stored in an aerosol in separate sealed packages and only combined upon use of the aerosol. The invention, which is straightforward to use, is intended for inpatient and private usage.

The invention relates to a pharmaceutical composition for nasal application in which the substances act on the central nervous system, and to adjuvant substances that act on the structures of the nasal mucous membranes and are predetermined for actively influencing the regulatory centers of the brain.

This invention is intended for in-patient and private use and is also not complicated to use.

Many biologically active molecules from the external environment and from the organism's internal environment are beneficial and necessary for maintaining the physiological functions of the organs and tissues, including the brain and the central nervous system (CNS) as a whole. Among these, for instance, are the majority of drugs and many biochemical compounds—metabolism products that occur in the organism due to the metabolism.

Delivering many known and novel drugs and biotechnology products, as well as a therapeutic application of various metabolites for treating the CNS and in particular diseases of the brain, is the serious problem.

For penetrating into the structures of the brain, the biologically active molecules, including regulatory metabolites, should penetrate into the structures of the brain through the plurality of biological membranes, of which the complexes of the blood-brain barrier are the least penetrable.

Nasal methods for delivering a few centrally acting drugs to the brain from the nasal cavity are described in the known scientific and patent literature of the last decade. The work of Ming Ming Wen (2011) describes the delivery of drug substances from the nasal cavity to the brain using various methods, in particular an application of mucoadhesive adjuncts that intensify the contact of the drug with the nasal mucosa, and the use of penetration enhancers, liposomes, nanoparticles, and other methods (Ming Ming Wen (2011) Intranasal delivery—physicochemical and therapeutic aspects. International Journal of Pharmaceutics 337, 1-24).

Although the suggested methods permit small molecules of the drugs to penetrate into the brain, for a broad spectrum of drug substances these methods are not universally suitable for delivering them to the brain.

Patent no. US 2005/0048002 A1 describes the manner in which drugs are delivered to the brain. For delivering drugs to the brain, the authors use cells that are loaded with pharmaceutical substances and thus are able to transport the drug from the blood to the brain. It is a drawback that the process for preparing the drug comprises comminution of the pharmaceutical substance into particles having an average size of approximately 150 nanometers to 100 micrometers and subsequently introducing the particles into the cells that are able to penetrate into the brain. Leukocytes, such as for instance macrophages, monocytes, granulocytes, and neutrophils, for instance, are used to this end. The leukocytes loaded with the drugs are introduced into the peripheral blood vessels intrathecally, intracerebrally, or epidurally. The manner described is technically complicated, invasive, and of limited use in clinical practice.

Known nasally introduced drugs reach the brain from the nasal cavity due both to the anatomical connections of the elements of the olfactory system and of the trigeminal nerve. Nevertheless, regardless of the direct relationship of the peripheral elements of the olfactory system and the brain, the efficacy of delivering drugs introduced nasally to the brain is very limited, which is associated in particular with the limited surface area of the olfactory epithelium, which forms less than 8% of the surface area of the nasal cavity's mucosa.

A modern approach to solving the problem of delivering drugs to the brain is the use of a few low molecular weight free radical substances. Inhalative application of active oxygen radicals for increasing the antinociceptive effects of analgesics that are introduced into the organism using a traditional path, such as orally or using an injection, is known (Goldstein, N., Lewin, T., Kamensky, A., Dubinin V., Baumann, S., Konstantinova, O. (1996) Exogenous gaseous superoxide potentiates the antinociceptive effect of opioid analgesic agents. Inflamm. Res., 5, 473-478).

Patent DE 19514 522 describes a pharmaceutical agent for treating pain that comprises an analgesic for treating strong pain and oxygen radicals and/or their secondary or decomposition products. One drawback of patent DE 195 14 522 is its limited therapeutic efficacy. Another drawback is comprised in its limited clinical applicability, which is related to the difficulties of a separate oral or injection administration of a drug substance and nasal introduction of SAR.

Patent RU no. 2253461 describes increasing the therapeutic efficacy of drug substances using a pharmaceutical combination in which the function of the CNS is influenced, wherein at least one substance is included that has a healing effect on the CNS and that is distinguished in that as the substance facilitating penetration of the blood-brain barrier acts by means of reflectory (preferably neuro- and vasodilatory) effects on the structures and receptors of the nasal cavity's mucosa, primarily on receptors of the vomeronasal organ and trigeminal nerve. However, experience has demonstrated that increasing the efficacy of the drugs is very different for different biologic substances in the pharmaceutical combination described in RU no. 2253461, which substantially complicates practical application.

Eurasian patent no. 010692 describes the pharmaceutical product for nasal administration for treating diseases and disorders of the central nervous system in which a mixture is used that contains the biologically active substances that act on the central nervous system, and those substances that act on the nasal mucous membrane, wherein the oxygen anion radicals and/or the active products of the nitrogen oxide are used. Despite certain advantages, there is a drawback in that the administration of said spray (see Item 14 of the formula) comprises a mixture of biologically active substances that act on the central nervous system and substances that act on the nasal mucous membrane such as oxygen anion radicals and/or active products of the nitrogen oxide.

Significant drawbacks of the spray in accordance with Eurasian patent no. 010692 are the uncontrollable interaction processes of the described mixture components, which reduce the expected efficacy of the drugs, metabolites, and/or other types of active substances described in the foregoing that are used and that are for treating the disorders of the central nervous system. Such a reduction in efficacy is not an obvious negative result of the administration of the spray comprising the mixture of the described and/or similar composition, the separate components thereof not only acting on different receptors, but also having an unexpected cross interaction.

The object of the invention is to increase the efficacy of the drugs, metabolites, and/or other types of active substances described in the foregoing that are used to treat diseases and disorders of the central nervous system and to lower the normally used dosage of the drugs, as well as to attain a more rapid and prolonged effect.

In attaining this object, potentiation of a combined composition of free radical substances and the biologically active substances during nasal administration is attained, wherein the combination of oxygen anion radicals (SAR) and active products of the nitrogen oxide (NO products) is realized when the substances are combined from separate chambers in an aerosol.

In the provided description, substances shall be construed to mean one or a plurality of pharmaceutical substances, metabolites, and/or types of biologically active substances described in the foregoing that treat diseases and disorders of the CNS.

Adjuvants shall be construed to mean individually or together with the other active oxygen radicals (SAR) and their reaction or other administered products such as superoxide ion (O₂), singlet oxygen (¹O₂), hydroxyl radical (HO*), perhydroxyl radical (HO₂*), hydrogen peroxide (H₂O₂), individually or together with nitrogen oxide sources (NO products) such as the sources of the active nitrogen oxide (NO), L-arginine, isosorbite mononitrate, nitroprusside, pentaerythril-tetranitrate, and glycerol tetranitrate, but not limited to them.

The invention shall be described in the following using examples.

The pharmaceutical composition described in the invention includes biologically active substances that must lead to creation of biologically active substances and metabolites, for instance from the classes of agonists of dopaminergic receptors, narcotic and non-narcotic analgesics, anti-epileptic drugs from the group of barbiturates, and an adjuvant containing free radical forms of anion radicals of oxygen or reaction products thereof (ROS), for instance hydrogen peroxide, and pharmacologically acceptable active products of nitrogen oxide (NO products).

The following examples demonstrate the increase in efficacy of biologically active substances that act on the central nervous system, in cooperation with substances that act on the nasal mucous membrane. The substances are each filled in separate sealed packaging and these substances are not combined from the packaging until administration.

Increasing the efficacy shall be construed to mean accelerating, enhancing, prolonging the duration of action, and/or potentiating the therapeutic effect, reducing therapeutic dosing, and reducing adverse reactions. The index of increase in efficacy is calculated using the formula:

$$Index ( IEE ) ( in \% ) = \frac{\begin{matrix}( Value in the primary group ) minus \\ ( Value in the compared group )\end{matrix}}{( Value in the compared group )} \times 100$$

### EXAMPLE 1

The restoration of spontaneous motor activity in rats as the sum of the movements in the test “Open Field” in the experimental model of a catalepsy caused by a single intraperitoneal (i.p.) injection of haloperidol (0.25 mg/kg, “Ratiopharm,” Germany). Dopamine (0.125 mg/kg, Polfa S.A. (Poland) was administered nasally in 50 μl in the solution of the spray once in each nostril in combination with the oxygen anion radicals (SAR) and/or the active substances of the nitrogen oxide according to the present invention (Group IV, “Composition 1”). The experiments were performed on non-purebred male rats having a body mass of 250±37 g.

TABLE 1
The efficacy comparison of anti-cataleptic effect of dopamine-
containing pharmaceutical composition (“Composition 1”) with
the composition “Dopamine/hydrogen peroxide/L-arginine”
(Group IV), and of the dopamine-containing pharmaceutical agent
according to Eurasian patent no. 010692 (Group III). The attribute
being investigated is the duration of the catalepsy caused by
intraperitoneal (i.p.) injection of haloperidol.

| Animal Group | Spontaneous Activity |
|---|---|
| I. Intact control (n = 6) | 36 ± 9 |
| II. Haloperidol (n = 6) | 3.6 ± 3.4 |
| III. Haloperidol + | 32 ± 6 |
| dopamine in composition | |
| according to pat. no. | |
| 010692 (n = 8) | |
| IV. Haloperidol (i.p.) + | 35 ± 11 |
| Composition 1 (n = 8) | |

IEE (IV to III) = 9.3%

The higher efficacy of the administration of the composition defined herein of the adjuvant and the dopamine according to the formula is demonstrated in Example 1 (Table 1). The pharmaceutical agent according to Eurasian patent no. 010692 attained adequate efficacy with a cross interaction of the substances contained in the spray.

### EXAMPLE 2

In clinical observations of patients having rigid and akinetic-rigid types of Parkinson's disease, the clinical advantages of the pharmaceutical composition of dopamine, SAR, and active products of nitrogen oxide (NO active) were attained when they were combined nasally from separate packages compared to introducing dopamine in the disadvantageous pharmaceutical form of the nasal spray according to Eurasian patent no. 010692 (Table 2).

All of the sick patients received a known therapy at the beginning of the treatment. Thereafter, the patients in “Primary group 1” received the inventive dopamine-containing pharmaceutical composition. The patients in “Primary group 2” received the dopamine-containing spray according to Eurasian patent no. 010692.

All patients in “Primary groups 1 and 2” received a treatment according to the same regime, specifically 2 times per day over 10 days. The patients in the “Control” group received only the known therapy. There were 3 patients in each group. The individual dosage of dopamine in “Primary groups 1 and 2” was 1.5 mg; the volume of a single introduced medication was 0.25 mL.

TABLE 2
Comparison of clinical results of nasal administration of pharmaceutical
composition in patients having rigid and akinetic-rigid types
of Parkinson's disease, comprising one biologically active
substance, dopamine, and one adjuvant, comprising SAR and the
active substance of nitrogen oxide, with its separated introduction
in the form of a spray, which substances are disposed in separate,
hermetically sealed packaging, and nasal introduction of dopamine
in the pharmaceutical form of the nasal spray according to Eurasian
patent no. 010692.

| | Patients with rigid | | Patients with akinetic rigid | |
|---|---|---|---|---|
| | type Parkinson's | | type Parkinson's | |
| | Assessment according | | Assessment according | |
| | to UPDRS scale | | to UPDRS scale | |
| | M ± σ | | M ± σ | |
| Patient group | Visit 1 | Visit 2 | Visit 1 | Visit 2 |
| I. Primary | 57.1 ± 16.5 | 17.9 ± 6.6 | 54.3 ± 12.4 | 25.3 ± 10.3 |
| group 1 | | | | |
| II. Primary | 55.8 ± 15.3 | 37.1 ± 16.9 | 55.6 ± 12.9 | 39.2 ± 17.1 |
| group 2 | | | | |
| III. Control | 57.8 ± 15.8 | 51.8 ± 19.0 | 56.4 ± 16.8 | 54.8 ± 14.9 |
| | IEE in % | | IEE in % | |
| | I to II | [52%] | I to II | [35%] |
| | (Visit 2) | | (Visit 2) | |

Terms: “Visit 1” = day of beginning of treatment; “Visit 2” = day treatment concluded [abs. value].

The observations described above demonstrate a higher therapeutic efficacy for the dopamine-containing pharmaceutical composition in the patients in “Primary group 1,” compared to the spray described in Eurasian patent no. 010692.

### EXAMPLE 3

The efficacy comparison of an analgesic action of a pharmaceutical composition according to the inventive patent application containing the analgesic trimeperidine in the Randall-Selitto pain test in rats. The analgesic was introduced nasally, once in each nostril, in a threshold dose of 1 mg/kg in a volume of 50 μL in the solution of the spray in the composition with the adjuvant, contains oxygen anion radicals (SAR) and/or the active products of the nitrogen oxide (NO active) (“Composition 2”, Group III), or in the composition of the spray according to Eurasian patent no. 010692 (Group II) (Table 3). The experiments were conducted on male, non-purebred white rats having a body weight of 220±35 g.

TABLE 3
The enhancement and prolonging of analgesic effect of the biologically active substance,
the analgesic trimeperidine, in the composition “Combination 2” (“trimeperidine/hydrogen
peroxide/L-arginine”) introduced nasally. The attributes investigated derive from
the intensity of critical pressure (ICP) on the rear paw of the rats and the duration
of the analgesic effect. The number of animals in each group = 10.

| | ICP base | ICP value (M ± σ) | | | | |
|---|---|---|---|---|---|---|
| | value | Time after administering composition, in minutes | | | | |
| Animal group | M ± σ | 5 | 15 | 60 | 180 | 240 |
| I. Intact control | 5.5 ± 0.8 | 5.2 ± 0.8 | 4.9 ± 0.8 | 5 ± 1.0 | 4.7 ± 0.9 | 5 ± 0.7 |
| II. Trimeperidine | 5.7 ± 0.4 | 7.2 ± 2.6 | 7.4 ± 2.1 | 7.9 ± 2.9 | 6.4 ± 2.4 | 6.1 ± 2.6 |
| according to | | | | | | |
| pat. no. 010692 | | | | | | |
| III. Trimeperidine in | 5.7 ± 0.4 | 9.2 ± 2.6 | 9.2 ± 2.9 | 8.9 ± 2.9 | 9.2 ± 2.2 | 8.4 ± 2.8 |
| “Composition 2” | | | | | | |

| | | |
|---|---|---|
| IEE in % | 5th minute | 240th minute |
| | (IV to III) | (IV K III) |
| | 27.8 | 37.7 |

It may be seen from Table 3 that the administration of the pharmaceutical “Composition 2” (Group III) in the critical times at the 5th and 240th minutes indicates an enhanced analgesic action compared to Group II in accordance with patent no. 010692. The duration of this effect also reflects prolongation of the analgesic effect at the 240th minute.

The positive effects of declared pharmaceutical “Composition 2” also apply for the substances that are known to penetrate the brain well in the usual manners of administration. In this case the increase in efficacy is revealed in the significant reduction in the dosage of biologically active substances (Example 4).

### EXAMPLE 4

The enhancement of the narcotic effect of the barbiturate phenobarbital in “Composition 3,” administered nasally.

TABLE 4
Narcotic action of phenobarbital in the pharmaceutical composition
“phenobarbital/hydrogen peroxide/L-arginine” (Group
II) compared to the result of nasal administration of a similar
dose of phenobarbital in accordance with patent no. 010692
(Group III). The number of animals in each group = 6.

| | Duration of sleep, in |
|---|---|
| Animal Group | minutes |
| I. Phenobarbital control | 192 ± 29.9 |
| 150 mg/kg | |
| II. Phenobarbital, 12.5 | 332 ± 41.9 |
| mg/kg in “Composition 3” | |
| III. Phenobarbital, 12.5 | 443 ± 56.5 |
| mg/kg in “Composition 3” | |
| IEE in % | 33.4 (III to II) |

The examples presented here demonstrate the increased efficacy and significant reduction in normally used dosages of drugs and metabolites and/or other types of biologically active substances that are used to treat diseases and disorders of the central nervous system and for attaining more rapid and prolonged action thereof.

1. A pharmaceutical composition for nasal administration in the treatment of diseases and disorders of the central nervous system in the drug form for nasal administration having drug substances that act on the central nervous system and substances that act on the nasal mucous membrane, in the form of oxygen anion radicals and/or active products of nitrogen oxides, wherein the biologically active substances that act on the central nervous system and the substances that act on the nasal mucous membrane are filled and stored in an aerosol in separate sealed packaging and are not combined until the aerosol is administered.

2. The pharmaceutical composition of claim 1, wherein the concentration of the substances acting on the nasal mucous membrane is from 10⁻⁸ to 10⁻¹ mol/L.

3. The pharmaceutical composition of claim 1, wherein the drug substances that act on the central nervous system are in the form of a spray.

4. The pharmaceutical composition of claim 1, wherein the drug substances that act on the central nervous system and the substances that act on the nasal mucous membrane are in the form of a spray.

5. The pharmaceutical composition of claim 1, wherein pharmaceutical substances that comprise a mixture with active products that act on the nasal mucous membrane, sources of nitrogen oxides that act on the central nervous system, in a concentration of 10⁻⁶ to 10⁻¹ mol/L, and are disposed in the form of an aerosol in a separate sealed packaging.

6. The pharmaceutical composition of claim 1, wherein the drug substances that act on the central nervous system are disposed in the mixture with the active substances from the sources of nitrogen oxides in the form of a spray.

7. The pharmaceutical composition of claim 1, wherein the drug substances that act on the central nervous system, in the mixture with the active substances of the nitrogen oxides, and the substances that act on the nasal mucous membrane, are disposed in the form of a spray.

8. The pharmaceutical composition of claim 1, wherein the substances that act on the nasal mucous membrane are disposed in the form of a spray.

9. The pharmaceutical composition of claim 2, wherein the concentration of the substances acting on the nasal mucous membrane is from 10⁻⁵ to 10⁻¹ mol/L.
